SEQUENCE LISTING (12 sequences)

SEQ ID NO: 1 - protein, 384 aa, Homo sapiens
MDPAGGPRGVLPRPCRVLVLLNPRGGKGKALQLFRSHVQPLLAEAEISFTLMLTERRNHARELVRSEELGRWDALVVMSGDGLMHEVVNGLMERPDWETAIQKPLCSLPAGSGNALAASLNHYAGYEQVTNEDLLTNCTLLLCRRLLSPMNLLSLHTASGLRLFSVLSLAWGFIADVDLESEKYRRLGEMRFTLGTFLRLAALRTYRGRLAYLPVGRVGSKTPASPVVVQQGPVDAHLVPLEEPVPSHWTVVPDEDFVLVLALLHSHLGSEMFAAPMGRCAAGVMHLFYVRAGVSRAMLLRLFLAMEKGRHMEYECPYLVYVPVVAFRLEPKDGKGVFAVDGELMVSEAVQGQVHPNYFWMVSGCVEPPPSWKPQQMPPPEEPL

SEQ ID NO: 2 - protein, 628 aa, Artificial Sequence
  It is synthesized
MDPAGGPRGVLPRPCRVLVLLNPRGGKGKALQLFRSHVQPLLAEAEISFTLMLTERRNHARELVRSEELGRWDALVVMSGDGLMHEVVNGLMERPDWETAIQKPLCSLPAGSGNALAASLNHYAGYEQVTNEDLLTNCTLLLCRRLLSPMNLLSLHTASGLRLFSVLSLAWGFIADVDLESEKYRRLGEMRFTLGTFLRLAALRTYRGRLAYLPVGRVGSKTPASPVVVQQGPVDAHLVPLEEPVPSHWTVVPDEDFVLVLALLHSHLGSEMFAAPMGRCAAGVMHLFYVRAGVSRAMLLRLFLAMEKGRHMEYECPYLVYVPVVAFRLEPKDGKGVFAVDGELMVSEAVQGQVHPNYFWMVSGCVEPPPSWKPQQMPPPEEPLGGGGSGGGGSGGGGSESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 3 - DNA, 1884 bp, Artificial Sequence
  It is synthesized
atggacccagcgggcggcccccggggcgtgctcccgcggccctgccgcgtgctggtgctgctgaacccgcgcggcggcaagggcaaggccttgcagctcttccggagtcacgtgcagccccttttggctgaggctgaaatctccttcacgctgatgctcactgagcggcggaaccacgcgcgggagctggtgcggtcggaggagctgggccgctgggacgctctggtggtcatgtctggagacgggctgatgcacgaggtggtgaacgggctcatggagcggcctgactgggagaccgccatccagaagcccctgtgtagcctcccagcaggctctggcaacgcgctggcagcttccttgaaccattatgctggctatgagcaggtcaccaatgaagacctcctgaccaactgcacgctattgctgtgccgccggctgctgtcacccatgaacctgctgtctctgcacacggcttcggggctgcgcctcttctctgtgctcagcctggcctggggcttcattgctgatgtggacctagagagtgagaagtatcggcgtctgggggagatgcgcttcactctgggcaccttcctgcgtctggcagccctgcgcacctaccgcggccgactggcttacctccctgtaggaagagtgggttccaagacacctgcctcccccgttgtggtccagcagggcccggtagatgcacaccttgtgccactggaggagccagtgccctctcactggacagtggtgcccgacgaggactttgtgctagtcctggcactgctgcactcgcacctgggcagtgagatgtttgctgcacccatgggccgctgtgcagctggcgtcatgcatctgttctacgtgcgggcgggagtgtctcgtgccatgctgctgcgcctcttcctggccatggagaagggcaggcatatggagtatgaatgcccctacttggtatatgtgcccgtggtcgccttccgcttggagcccaaggatgggaaaggtgtgtttgcagtggatggggaattgatggttagcgaggccgtgcagggccaggtgcacccaaactacttctggatggtcagcggttgcgtggagcccccgcccagctggaagccccagcagatgccaccgccagaagagcccttaggcggaggcggaagcggaggcggaggaagcggcggtggcggcagcgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaa

SEQ ID NO: 4 - protein, 20 aa, Artificial Sequence
  It is synthesized
MYRMQLLSCIALSLALVTNS

SEQ ID NO: 5 - DNA, 1152 bp, Artificial Sequence
  It is synthesized
atggacccagcgggcggcccccggggcgtgctcccgcggccctgccgcgtgctggtgctgctgaacccgcgcggcggcaagggcaaggccttgcagctcttccggagtcacgtgcagccccttttggctgaggctgaaatctccttcacgctgatgctcactgagcggcggaaccacgcgcgggagctggtgcggtcggaggagctgggccgctgggacgctctggtggtcatgtctggagacgggctgatgcacgaggtggtgaacgggctcatggagcggcctgactgggagaccgccatccagaagcccctgtgtagcctcccagcaggctctggcaacgcgctggcagcttccttgaaccattatgctggctatgagcaggtcaccaatgaagacctcctgaccaactgcacgctattgctgtgccgccggctgctgtcacccatgaacctgctgtctctgcacacggcttcggggctgcgcctcttctctgtgctcagcctggcctggggcttcattgctgatgtggacctagagagtgagaagtatcggcgtctgggggagatgcgcttcactctgggcaccttcctgcgtctggcagccctgcgcacctaccgcggccgactggcttacctccctgtaggaagagtgggttccaagacacctgcctcccccgttgtggtccagcagggcccggtagatgcacaccttgtgccactggaggagccagtgccctctcactggacagtggtgcccgacgaggactttgtgctagtcctggcactgctgcactcgcacctgggcagtgagatgtttgctgcacccatgggccgctgtgcagctggcgtcatgcatctgttctacgtgcgggcgggagtgtctcgtgccatgctgctgcgcctcttcctggccatggagaagggcaggcatatggagtatgaatgcccctacttggtatatgtgcccgtggtcgccttccgcttggagcccaaggatgggaaaggtgtgtttgcagtggatggggaattgatggttagcgaggccgtgcagggccaggtgcacccaaactacttctggatggtcagcggttgcgtggagcccccgcccagctggaagccccagcagatgccaccgccagaagagccctta

SEQ ID NO: 6 - DNA, 57 bp, Artificial Sequence
  It is synthesized
ctccatagaagattctagagctagggatccgccaccatgtacaggatgcaactcctg

SEQ ID NO: 7 - DNA, 41 bp, Artificial Sequence
  It is synthesized
gggccgcccgctgggtccatcgaattcgtgacaagtgcaag

SEQ ID NO: 8 - DNA, 19 bp, Artificial Sequence
  It is synthesized
atggacccagcgggcggcc

SEQ ID NO: 9 - DNA, 59 bp, Artificial Sequence
  It is synthesized
gccaccgccgcttcctccgcctccgcttccgcctccgcctaagggctcttctggcggtg

SEQ ID NO: 10 - DNA, 59 bp, Artificial Sequence
  It is synthesized
gcggaggaagcggcggtggcggcagcgagtccaaatatggtcccccatgcccatcatgc

SEQ ID NO: 11 - DNA, 44 bp, Artificial Sequence
  It is synthesized
gtaatccagaggttgattgtcgactcatttacccggagacaggg

SEQ ID NO: 12 - protein, 229 aa, Artificial Sequence
  It is synthesized
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

(19) United States
(12) Patent Application Publication
(10) Pub. No.: US 2021/0388326 A1
(43) Pub. Date: Dec. 16, 2021

(54) SPHINGOSINE KINASE 1 AND FUSION PROTEIN COMPRISING THE SAME AND USE THEREOF

(71) Applicants: Haifeng DUAN, Beijing (CN); BEIJING SHUANGYIN BIOTECHNOLOGY CO. LTD., Beijing (CN)

(72) Inventors: Haifeng DUAN, Beijing (CN); Jing XIE, Beijing (CN); Xianwen HU, Beijing (CN); Jingbo GONG, Beijing (CN); Binghua XUE, Beijing (CN); Qunwei ZHANG, Beijing (CN); Xiuxiao XIAO, Beijing (CN); Meilan CUI, Beijing (CN); Rumeng PANG, Beijing (CN); Rui WANG, Beijing (CN); Tingting YU, Beijing (CN)

(21) Appl. No.: 17/286,445

(22) PCT Filed: Sep. 20, 2019
(86) PCT No.: PCT/CN2019/107091

§ 371 Date: Apr. 16, 2021

(30) Foreign Application Priority Data
Oct. 21, 2018 (CN) 201811225720.2

(51) Int. Cl. C12N 9/12; A61K 38/20; C07K 14/55; A61K 38/45; A61K 47/60; C12N 15/86; A61P 3/10

(52) U.S. Cl. CPC C12N 9/1205; A61K 38/2013; C07K 14/55; A61K 38/45; C12Y 207/01091; C12N 2740/15043; C12N 15/86; A61P 3/10; C07K 2319/30; C07K 2319/02; A61K 47/60

(57) ABSTRACT

The present invention provides a sphingosine kinase 1, a fusion protein comprising the same, and a use thereof. The sphingosine kinase 1 and the fusion protein comprising the same have significant effects in lowering blood sugar and body weight, and are useful for the preparations of protein drugs for controlling metabolic diseases such as obesity and diabetes. It also provides a protein drug, which is a fusion protein containing a sphingosine kinase 1 or an amino acid sequence having the activity thereof. The fusion protein comprises a sphingosine kinase 1 (SPHK1) or an amino acid sequence having the activity thereof, a FC sequence and a linker sequence. The protein drug can significantly decrease blood sugar, blood fat and body weight, and improve fat metabolism.

### TECHNICAL FIELD

The invention relates to the field of biopharmaceuticals. Specifically, the present invention relates to a use of sphingosine kinase 1, more specifically, relates to a fusion protein of sphingosine kinase 1, in particular to a fusion protein containing sphingosine kinase 1 and FC sequences and its use.

### BACKGROUND ART

Obesity and type 2 diabetes (T2DM) are among the major public health problems that plague modern society. Obesity and its accompanying insulin resistance are key factors in the onset of type 2 diabetes. It has been reported that 80-90% of type 2 diabetes patients are overweight or obese (Zou Dajin et al., Shanghai Medical, 2014, 37 (9), 729-734). Therefore, effective control of blood sugar and body weight has always been a focus topic in the related art. According to statistics, there are currently 400 million people with type 2 diabetes worldwide, accounting for 90%-95% of all diabetes patients. The drugs currently used to treat diabetes mainly include insulin and oral hypoglycemic drugs such as metformin, but the shortcomings of these drugs are that they easily cause hypoglycemia and have no obvious effect on the control of the patient's weight. Another class of drugs are GLP-1 receptor agonist drugs, such as Liraglutide from Novo Nordisk and Dularutide from Eli Lilly. Such drugs can also reduce weight while controlling blood sugar, but it is mainly achieved by suppressing the patient's appetite and controlling the patient's food intake, which greatly reduces the patient's quality of life.

There is also a class of enzymes that play a very important role in regulating metabolism in the human body, such as sphingosine kinase 1 (SphK1). SphK1, as a recently discovered family of lipid kinases, are conserved in humans, mice, yeast and plants in view of evolution. This enzyme is believed a key enzyme in the metabolism pathway of sphingolipid, which catalyzes the formation of sphingosine-1-phosphate (S1P) from sphingosine, and is a “Rheostat” regulating the synthesis of ceramide and sphingosine-1-phosphate (S1P). SphK1 catalyzes the production of S1P from sphingosine, which is a metabolite of ceramide. After binding to receptors, S1P can regulate cell processes such as cell growth, apoptosis, differentiation and hematopoiesis. The SphK1/S1P signaling pathway is involved in various biological processes and diseases, including tumorigenesis and diabetes. The prior art shows that SphK1 can be secreted out of the cell, but its role in the extracellular environment and the presence of extracellular receptors are unclear (Venkataraman K, et al. Biochemical Journal, 2006, 397 (3): 46 1-71). Furthermore, loss of SphK1 can promote pancreatic cell apoptosis in mice under a high-sugar and high-fat diet, thereby inducing the formation of diabetes (Qi Y, et al. Faseb Journal, 2013, 27 (10): 4294-4304). In addition, diabetic mice, after injection of adenovirus carrying the human SphK1 gene, showed reduced blood glucose and blood lipid levels compared to control mice. At present, the research on SphK1 is mainly based on its role in cells, so the drugs developed with this protein as the target mainly use its antibodies or antagonists, and the protein is not directly made into drugs for treatment. The other is to use the virus as a carrier to pour the SPHK1 gene into cells for gene therapy, such as gene therapy using adenovirus as a carrier. However, this method is easy to develop drug resistance in the body, and the treatment of metabolic diseases such as diabetes requires long-term medication, which limits the use of this method. Therefore, drug development based on human SPHK1 gene has broad prospects.

### SUMMARY OF THE INVENTION

Based on the above-mentioned problems of the prior art, one object of the present invention is to provide a use of sphingosine kinase 1. The inventor unexpectedly discovered that, when directly made into protein drugs, SphK1 can function outside the cell without entering the cell, and has a significant effect of lowering blood sugar and weight. Therefore, the present invention provides a use of sphingosine kinase 1 in the preparation of drugs for preventing and/or treating obesity, hyperlipidemia, or diabetes. The present invention also provides a protein drug containing a sphingosine kinase 1 (SPHK1), a method for preparing the protein drug and a use of the protein drug. Compared with the prior art, the protein drug according to the present invention can significantly reduce blood sugar, blood lipids, body weight and improve fat metabolism.

In one aspect, the present invention provides a use of a sphingosine kinase 1 or an amino acid sequence having the activity thereof in the preparation of protein drugs for preventing and/or treating obesity, hyperlipidemia or diabetes.

Preferably, the sphingosine kinase 1 or the amino acid sequence having the activity thereof comprises the amino acid sequence shown in SEQ ID NO: 1.

In another aspect, the present invention provides a protein drug containing a sphingosine kinase 1 or an amino acid sequence having the activity thereof.

Preferably, the protein drug is a fusion protein containing the sphingosine kinase 1 or the amino acid sequence having the activity thereof. More preferably, the fusion protein contains the sphingosine kinase 1 (SPHK1) or the amino acid sequence having the activity thereof, a FC sequence and a linker sequence.

The FC sequence is selected from the amino acid sequence of human or animal immunoglobulin and its subtypes and variants, or the amino acid sequence of human or animal albumin and its variants.

Preferably, the general formula of the linker sequence is (GGGGS)n, where n is an integer of 0-5, preferably, 3.

The human or animal immunoglobulin is preferably selected from IgG4FC fragments, more preferably, selected from the amino acid sequence shown in SEQ ID NO: 12.

Preferably, the fusion protein contains the amino acid sequence shown in SEQ ID NO: 2.

In one preferred embodiment, the fusion protein is modified with polyethylene glycol. The average molecular weight of the polyethylene glycol is preferably 5-50 KD, more preferably 20-45 KD. Preferably, the polyethylene glycol is a linear or branched polyethylene glycol.

In another aspect, the present invention provides a coding gene containing the coding nucleotide sequence of the protein drug as disclosed above, preferably, the coding nucleotide sequence as shown in SEQ ID NO: 3.

In yet another aspect, the present invention provides an expression construct containing the coding nucleotide sequence of the protein drug as disclosed above, preferably, the coding nucleotide sequence as shown in SEQ ID NO: 3.

Preferably, the expression construct is a prokaryotic expression construct. More preferably, the prokaryotic expression construct is a pET vector series.

Alternatively, the expression construct is a eukaryotic expression construct. Preferably, the eukaryotic expression construct is a plasmid DNA vector, preferably pVAX1 vector and pSV1.0 vector; a recombinant viral vector, preferably recombinant vaccinia virus vector, recombinant adenovirus Vector or recombinant adeno-associated viral vector; or a retroviral vector/lentiviral vector, preferably HIV viral vector.

In another aspect, the present invention provides a host cell comprising the expression construct as disclosed above.

Preferably, when the expression construct is a prokaryotic expression construct, the host cell is a prokaryotic cell, preferably bacterial cell; or when the expression construct is a eukaryotic expression construct, the host cell is true nuclear organism cells, preferably mammalian cells, more preferably CHO cells.

In another aspect, the present invention provides a method for preparing a protein drug, comprising the step of cloning the nucleotide sequence of the protein drug into an expression vector.

Specifically, the method comprises the following steps:

1) constructing the nucleic acid sequence of the above protein drug;

2) constructing an expression vector containing the nucleic acid sequence of step 1);

3) utilizing the expression vector of step 2) to transfect or transform a host cell and allow the nucleic acid sequence to be expressed in the host cell;

4) purifying the protein expressed in step 3).

Preferably, in step 3), the host cell is a CHO-S cell.

The present invention also provides a use of the above-mentioned protein drug, coding gene, expression construct, and host cell in the preparation of pharmaceutical compositions for preventing and/or treating obesity, hyperlipidemia, or diabetes.

Compared with the prior art, the present invention has the following advantages that: the sphingosine kinase 1 and the fusion protein containing the same as disclosed in the present invention have significant effects in lowering blood sugar and body weight, and can be used to prepare protein drugs for controlling metabolic diseases such as obesity and diabetes.

### DESCRIPTION OF FIGURES

Hereinafter, the embodiments of the present invention will be described in detail with reference to the drawings, in which:

FIG. 1 is a schematic diagram of the vector construction of pCDH-SPHK1-L-Fc according to the present invention;

FIG. 2 is the expression of protein SPHK1-Fc detected by Western blotting, where a is the expression of protein in the supernatant after lentivirus infection of cells, “Blank” is the supernatant of virus-infected cells, detected with human IgG4Fc specific antibody, b is the purified SDS-PAGE electrophoresis diagram;

FIG. 3 shows the effects of the SPHK1 protein and its fusion protein SPHK1-Fc of the present invention to the fasting blood glucose level in type II diabetic model mice, where the control is the saline group, and * represents a significant difference compared to the control (p value<0.05);

FIG. 4 shows the treatment effects of the SPHK1 protein and its fusion protein SPHK1-Fc according to the present invention after 2 weeks to the body weight of type II diabetes model mice, where the control is saline group, and * represents significant difference compared to the control (p value<0.05);

FIG. 5 shows the treatment effects of the SPHK1 protein and its fusion protein SPHK1-Fc according to the present invention to after 2 weeks to the glucose tolerance of type II diabetes model mice, where the control is saline group, * represents a significant difference compared to the control (p value<0.05), and ** represents a very significant difference compared to the control (p value<0.001);

FIG. 6 shows the treatment effects of the SPHK1 protein and its fusion protein SPHK1-Fc according to the present invention to the lipid level in type II diabetic model mice, where the control is saline group, and * represents a significant difference compared to the control (p value<0.05).

### EMBODIMENTS

The present invention will be further described in detail below through the embodiments and examples. Through these descriptions, the characteristics and advantages of the present invention will become clearer.

The term “exemplary” herein means “serving as an example, embodiment, or illustration.” Any embodiment described herein as “exemplary” need not be construed as being superior to or better than other embodiments.

Unless otherwise specified, the reagents used in the following examples are analytical grade reagents, and are commercially available.

### Example 1 Preparation of Fusion Protein SPHK1-Fc

1. Construction of Lentiviral Expression Vector pCDH-SPHK1-L-Fc Containing Fusion Protein SPHK1-Fc

Among them, the sphingosine kinase 1 or the amino acid sequence having the activity thereof includes, for example, SEQ ID NO: 1:

MDPAGGPRGVLPRPCRVLVLLNPRGGKGKALQLFRSHVQPLLAEAEISFT
LMLTERRNHARELVRSEELGRWDALVVMSGDGLMHEVVNGLMERPDWETA
IQKPLCSLPAGSGNALAASLNHYAGYEQVTNEDLLTNCTLLLCRRLLSPM
NLLSLHTASGLRLFSVLSLAWGFIADVDLESEKYRRLGEMRFTLGTFLRL
AALRTYRGRLAYLPVGRVGSKTPASPVVVQQGPVDAHLVPLEEPVPSHWT
VVPDEDFVLVLALLHSHLGSEMFAAPMGRCAAGVMHLFYVRAGVSRAMLL
RLFLAMEKGRHMEYECPYLVYVPVVAFRLEPKDGKGVFAVDGELMVSEAV
QGQVHPNYFWMVSGCVEPPPSWKPQQMPPPEEPL;

The coding nucleotide sequence is shown as SEQ ID NO: 5:

ATGGACCCAGCGGGCGGCCCCCGGGGCGTGCTCCCGCGGCCCTGCCGCGT
GCTGGTGCTGCTGAACCCGCGCGGCGGCAAGGGCAAGGCCTTGCAGCTCT
TCCGGAGTCACGTGCAGCCCCTTTTGGCTGAGGCTGAAATCTCCTTCACG
CTGATGCTCACTGAGCGGCGGAACCACGCGCGGGAGCTGGTGCGGTCGGA
GGAGCTGGGCCGCTGGGACGCTCTGGTGGTCATGTCTGGAGACGGGCTGA
TGCACGAGGTGGTGAACGGGCTCATGGAGCGGCCTGACTGGGAGACCGCC
ATCCAGAAGCCCCTGTGTAGCCTCCCAGCAGGCTCTGGCAACGCGCTGGC
AGCTTCCTTGAACCATTATGCTGGCTATGAGCAGGTCACCAATGAAGACC
TCCTGACCAACTGCACGCTATTGCTGTGCCGCCGGCTGCTGTCACCCATG
AACCTGCTGTCTCTGCACACGGCTTCGGGGCTGCGCCTCTTCTCTGTGCT
CAGCCTGGCCTGGGGCTTCATTGCTGATGTGGACCTAGAGAGTGAGAAGT
ATCGGCGTCTGGGGGAGATGCGCTTCACTCTGGGCACCTTCCTGCGTCTG
GCAGCCCTGCGCACCTACCGCGGCCGACTGGCTTACCTCCCTGTAGGAAG
AGTGGGTTCCAAGACACCTGCCTCCCCCGTTGTGGTCCAGCAGGGCCCGG
TAGATGCACACCTTGTGCCACTGGAGGAGCCAGTGCCCTCTCACTGGACA
GTGGTGCCCGACGAGGACTTTGTGCTAGTCCTGGCACTGCTGCACTCGCA
CCTGGGCAGTGAGATGTTTGCTGCACCCATGGGCCGCTGTGCAGCTGGCG
TCATGCATCTGTTCTACGTGCGGGCGGGAGTGTCTCGTGCCATGCTGCTG
CGCCTCTTCCTGGCCATGGAGAAGGGCAGGCATATGGAGTATGAATGCCC
CTACTTGGTATATGTGCCCGTGGTCGCCTTCCGCTTGGAGCCCAAGGATG
GGAAAGGTGTGTTTGCAGTGGATGGGGAATTGATGGTTAGCGAGGCCGTG
CAGGGCCAGGTGCACCCAAACTACTTCTGGATGGTCAGCGGTTGCGTGGA
GCCCCCGCCCAGCTGGAAGCCCCAGCAGATGCCACCGCCAGAAGAGCCCT
TA

The amino acid sequence of the fusion protein SPHK1-Fc is shown as SEQ ID NO: 2, and its nucleotide sequence is shown as SEQ ID NO: 3. Its N segment to C segment are SPHK1, linkrt sequence L and Fc in sequence;

The amino acid sequence of Fc is shown as SEQ ID NO: 12:

ESKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSPGK.

The amino acid sequence of the fusion protein SPHK1-Fc is shown as SEQ ID NO: 2, where the bold italic part is the amino acid sequence of the linker sequence, and the underlined part is the amino acid sequence of Fc:

MDPAGGPRGVLPRPCRVLVLLNPRGGKGKALQLFRSHVQPLLAEAEISFT
LMLTERRNHARELVRSEELGRWDALVVMSGDGLMHEVVNGLMERPDWETA
IQKPLCSLPAGSGNALAASLNHYAGYEQVTNEDLLTNCTLLLCRRLLSPM
NLLSLHTASGLRLFSVLSLAWGFIADVDLESEKYRRLGEMRFTLGTFLRL
AALRTYRGRLAYLPVGRVGSKTPASPVVVQQGPVDAHLVPLEEPVPSHWT
VVPDEDFVLVLALLHSHLGSEMFAAPMGRCAAGVMHLFYVRAGVSRAMLL
RLFLAMEKGRHMEYECPYLVYVPVVAFRLEPKDGKGVFAVDGELMVSEAV
QGQVHPNYFWMVSGCVEPPPSWKPQQMPPPEEPLGGGGSGGGGSGGGGSE
SKYGPPCPSCPAPEFLGGPSVFLFPPKPKDTLMISRTPEVTCVVVDVSQE
DPEVQFNWYVDGVEVHNAKTKPREEQFNSTYRVVSVLTVLHQDWLNGKEY
KCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQE
GNVFSCSVMHEALHNHYTQKSLSLSPGK.

The nucleotide sequence is shown as SEQ ID NO: 3, where the italic part is the nucleotide sequence of the linker sequence, and the underlined part is the nucleotide sequence of Fc:

ATGGACCCAGCGGGCGGCCCCCGGGGCGTGCTCCCGCGGCCCTGCCGCGT
GCTGGTGCTGCTGAACCCGCGCGGCGGCAAGGGCAAGGCCTTGCAGCTCT
TCCGGAGTCACGTGCAGCCCCTTTTGGCTGAGGCTGAAATCTCCTTCACG
CTGATGCTCACTGAGCGGCGGAACCACGCGCGGGAGCTGGTGCGGTCGGA
GGAGCTGGGCCGCTGGGACGCTCTGGTGGTCATGTCTGGAGACGGGCTGA
TGCACGAGGTGGTGAACGGGCTCATGGAGCGGCCTGACTGGGAGACCGCC
ATCCAGAAGCCCCTGTGTAGCCTCCCAGCAGGCTCTGGCAACGCGCTGGC
AGCTTCCTTGAACCATTATGCTGGCTATGAGCAGGTCACCAATGAAGACC
TCCTGACCAACTGCACGCTATTGCTGTGCCGCCGGCTGCTGTCACCCATG
AACCTGCTGTCTCTGCACACGGCTTCGGGGCTGCGCCTCTTCTCTGTGCT
CAGCCTGGCCTGGGGCTTCATTGCTGATGTGGACCTAGAGAGTGAGAAGT
ATCGGCGTCTGGGGGAGATGCGCTTCACTCTGGGCACCTTCCTGCGTCTG
GCAGCCCTGCGCACCTACCGCGGCCGACTGGCTTACCTCCCTGTAGGAAG
AGTGGGTTCCAAGACACCTGCCTCCCCCGTTGTGGTCCAGCAGGGCCCGG
TAGATGCACACCTTGTGCCACTGGAGGAGCCAGTGCCCTCTCACTGGACA
GTGGTGCCCGACGAGGACTTTGTGCTAGTCCTGGCACTGCTGCACTCGCA
CCTGGGCAGTGAGATGTTTGCTGCACCCATGGGCCGCTGTGCAGCTGGCG
TCATGCATCTGTTCTACGTGCGGGCGGGAGTGTCTCGTGCCATGCTGCTG
CGCCTCTTCCTGGCCATGGAGAAGGGCAGGCATATGGAGTATGAATGCCC
CTACTTGGTATATGTGCCCGTGGTCGCCTTCCGCTTGGAGCCCAAGGATG
GGAAAGGTGTGTTTGCAGTGGATGGGGAATTGATGGTTAGCGAGGCCGTG
CAGGGCCAGGTGCACCCAAACTACTTCTGGATGGTCAGCGGTTGCGTGGA
GCCCCCGCCCAGCTGGAAGCCCCAGCAGATGCCACCGCCAGAAGAGCCCT
TAGGCGGAGGCGGAAGCGGAGGCGGAGGAAGCGGCGGTGGCGGCAGCGAG
TCCAAATATGGTCCCCCATGCCCATCATGCCCAGCACCTGAGTTCCTGGG
GGGACCATCAGTCTTCCTGTTCCCCCCAAAACCCAAGGACACTCTCATGA
TCTCCCGGACCCCTGAGGTCACGTGCGTGGTGGTGGACGTGAGCCAGGAA
GACCCCGAGGTCCAGTTCAACTGGTACGTGGATGGCGTGGAGGTGCATAA
TGCCAAGACAAAGCCGCGGGAGGAGCAGTTCAACAGCACGTACCGTGTGG
TCAGCGTCCTCACCGTCCTGCACCAGGACTGGCTGAACGGCAAGGAGTAC
AAGTGCAAGGTCTCCAACAAAGGCCTCCCGTCCTCCATCGAGAAAACCAT
CTCCAAAGCCAAAGGGCAGCCCCGAGAGCCACAGGTGTACACCCTGCCCC
CATCCCAGGAGGAGATGACCAAGAACCAGGTCAGCCTGACCTGCCTGGTC
AAAGGCTTCTACCCCAGCGACATCGCCGTGGAGTGGGAGAGCAATGGGCA
GCCGGAGAACAACTACAAGACCACGCCTCCCGTGCTGGACTCCGACGGCT
CCTTCTTCCTCTACAGCAGGCTAACCGTGGACAAGAGCAGGTGGCAGGAG
GGGAATGTCTTCTCATGCTCCGTGATGCATGAGGCTCTGCACAACCACTA
CACACAGAAGAGCCTCTCCCTGTCTCCGGGTAAA.

The signal peptide sequence is the IL-2 signal peptide sequence (SP, the amino acid sequence is shown as SEQ ID NO:4; SEQ ID NO: 4MYRMQLLSCIALSLALVTNS). Primers are designed based on the IL-2 signal peptide, including armSP-F (upstream primer)(SEQ ID NO:6: CTCCATAGAAGATTCTAGAGCTAGGGATCCGCCACCATGTACAGGATGCAA CTCCTG) and armSP-R (downstream primer)(SEQ ID NO:7: GGGCCGCCCGCTGGGTCCATCGAATTCGTGACAAGTGCAAG), using the plasmid pFUSE-hIgG4-Fc2 (purchased from Dakco PCR Amplification Technology Co., Ltd.) as a template, PCR amplified fragment SP (116 bp). Primers are designed based on the nucleotide sequence of SPHK1 such as SEQ ID NO:5, including SPHK1-F (upstream primer)(SEQ ID NO:8: ATGGACCCAGCGGGCGGCC) and SPHK1-R (downstream primer)(SEQ ID NO:9:GCCACCGCCGCTTCCTCCGCCTCCGCTTCCGCCTCCGCCTAAGGGCT CTTCTGGCGGTG), using the plasmid pcDNA3.1-WSPK1c (Institute of Military Medical Sciences) as a template, PCR amplified fragment SPHK1 (1191 bp). The PCR product fragment 3′ segment contains a part of the linker sequence L (5′-GGCGGAGGCGGAAGCGGAGGCGGAGGAAGGC). Primers are designed based on the nucleotide sequence of Fc protein such as SEQ ID NO:3, including Fc-F (SEQ ID NO:10: GCGGAGGAAGCGGCGGTGGCGGCAGCGAGTCCAAATATGGTCCCCCATGC CC ATCATGC) and Fc-R (SEQ ID NO:11 GTAATCCAGAGGTTGATTGTCGACTCATTTACCCGGAGACAGGG), using the plasmid pFUSE-hIgG4-Fc2 (purchased from Daktronics Biotechnology Co., Ltd.) as a template, PCR amplified fragment Fc (740 bp). The PCR product fragment 5′ segment contains a part of the linker sequence L (sequence 5′-GCGGAGGAAGCGGCGGTGGCGGCAGC-3′). All primers were synthesized by Beijing Qingke Xinye Biotechnology Co., Ltd. The PCR reaction system and reaction conditions are shown in Table 1 and Table 2.

TABLE 1
PCR reaction system

| | | |
|---|---|---|
| 10 × ExTaq buffer (purchased from Takara Company, | 5 | μL |
| catalog number RR001A) | | |
| dNTP mixture (each 2.5 mM) | 4 | μL |
| (purchased from Takara, article number RR001A) | | |
| upstream primer (10 μM) | 2.5 | μL |
| downstream primer (10 μM) | 2.5 | μL |
| template (0.1 μg/μL) | 1 | μL |
| ExTaq (purchased from Takara, article number RR001A, | 0.25 | μL |
| 5 U/μL) | | |

| | |
|---|---|
| H₂O | make up to |
| | 50 μL |

TABLE 2
PCR reaction conditions

| 95° C. | 4 | min |
|---|---|---|
| 95° C. | 30 | s |
| annealing temperature * | 30 | s |
| 72° C. | extended time # | |
| 72° C. | 5 | min |

| | |
|---|---|
| 10° C. | forever |

PCR was performed for 30 cycles.
* Annealing temperature of different fragments amplified by PCR is Tm value of primer −3° C.;
# PCR extension time of different fragments is lkb/min.

After the reaction was completed, the obtained product was subjected to 1% agarose gel electrophoresis, and the PCR products (i.e. sp, SPHK1, and Fc) each individually were subjected to gel recovery purification (the gel recovery kit was purchased from Tiangen Company).

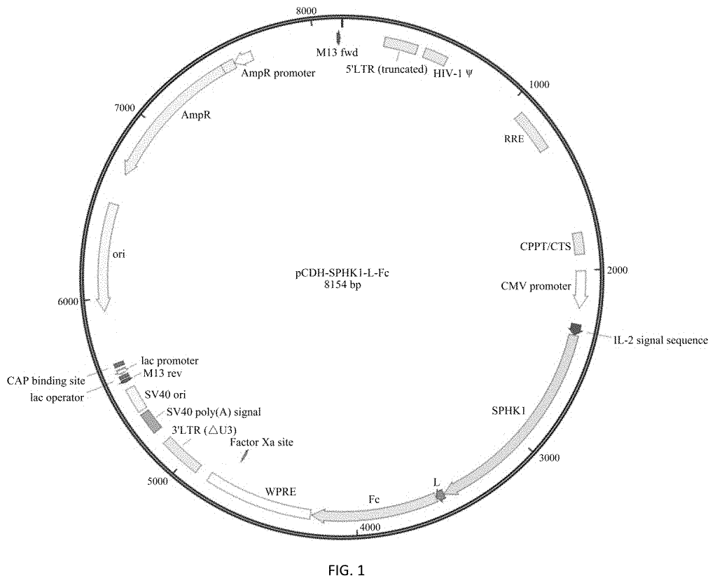

The plasmid pCDH-CMV (purchased from Addgene) was double digested with BamHI and SalI. The digested product was excised and recovered by gel, and then was ligated with the above purified PCR products (i.e. sp, SPHK1, and Fc) by streamless cloning (Streamless Assembly Cloning Kit, Purchased from Clone Smarter technologies). The ligation product was transformed into DH5a competent cells (purchased from Tiangen Biochemical Technology Co., Ltd.). For the transformation method, please refer to the instructions of competent cells. The transformed bacterial solution was coated on an LB plate containing 100 μg/mL ampicillin and incubated overnight at 37° C. Single clones were picked for colony PCR. The positive clones were sent to Beijing Qingke Xinye Biotechnology Co., Ltd. for sequencing. The clones with correct sequencing results were saved and extracted to obtain the plasmid, and the plasmid obtained was named pCDH-SPHK1-L-Fc. The plasmid map is shown in FIG. 1.

### Example 2 Preparation of Lentiviral Particles Carrying Plasmid pCDH-SPHK1-L-Fc

293T cells (Lab217 Embryo Engineering Laboratory of Northeast Agricultural University) with a cell confluence of over 90% were inoculated into a 150 mm petri dish (1.2×10⁷ cells per dish), 20 ml of DMEM medium containing 10% FBS was then added, and the cells were cultured under 37° C. and 5% CO₂ saturated humidity. 2 h before transfection, the original medium was removed and replaced by 18 ml of serum-free DMEM medium. The above-prepared p-SPHK1-L-Fc plasmid was mixed with the lentiviral packaged auxiliary plasmids pHelper1 and pHelper2 (Northeast Agricultural University Lab217 Embryo Engineering Laboratory) in equal proportions, respectively. 293T cells were transfected referring to the instructions of liposome Lipofectamin 2000 transfection kit (Purchased from Invitrogen). After 6 to 8 hours of transfection, the supernatant containing the transfection mixture was removed, 20 ml of fresh DMEM medium containing 5% FBS was added to each dish, and then the cells were cultured under 37° C. and 5% CO₂ saturated humidity. After 24 h, the supernatant was collected and stored at 4° C., and a fresh 20 ml medium was added. After another 24 hours of culture, the supernatant was collected once more. The supernatants collected two times were centrifuged at 4° C. and 3500 rpm for 15 min, and the pellet was removed. The supernatants each individually were centrifuged and concentrated with an Amicon Ultra-15 ultrafiltration tube (10 KD, purchased from Millipore) to obtain lentiviral particles carrying SPHK1-Fc, respectively. After the virus titer determination, the virus particles were diluted to 1×10⁸ TU/ml, packed separately and stored at −80° C.

### Example 3 Lentivirus Infection of CHO-S Cells and Screening and Verification of Positive Monoclonal

3.1 Lentivirus Infection of CHO-S Cells

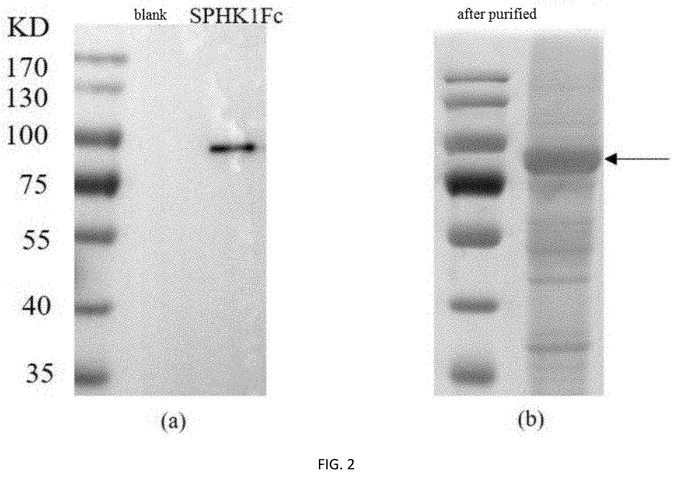

The suspended FreeStyle CHO-S cells (purchased from Thermo Scientific) were inoculated at 2×10⁵ cells/mL in 30 mL CD-SFM medium (CD FortiCHO medium+8 mM glutamine+1×HT supplement, all purchased from Thermo scientific company) in a 125 mL shake flask (purchased from Corning) under the conditions of 120 rpm, 8% CO₂, and 37° C. until logarithmic growth phase. Then, the cells were diluted with CD-SFM medium to 4×10⁴ cells/mL cell suspension. 0.5 mL of cell suspension was taken, mixed with the above lentiviral particles according to the multiplicity of infection (MOI) of 80, and then centrifuged at 800 g and 32° C. for 32 min. The supernatant was removed. The cells were resuspended by adding 0.5 mL CD-SFM medium and transferred to a 24-well plate. After cultured under 37° C. and 5% CO₂ for 48 to 72 hours, the expression of the target protein in the culture supernatant was detected by Western blot. After 30 μL of cell supernatant was taken and reduced to SDS-PAGE by 10%, the protein was transferred to the PVDF membrane by low-temperature wet method under the condition of constant current 300 mA for 1 h, and blocked with 5% skim milk/TBST solution at room temperature for 1 hour. The expression of SPHK1-Fc was detected with HRP-conjugated mouse anti-human IgG4Fc antibody (diluted at a ratio of 1:3000, purchased from abcam). The antibody was incubated at room temperature for 1 hour and washed with TBST solution 3 times (for 10 min each time). The ECL luminescence imaging system (purchased from Beijing Yuanpinghao Biotechnology Co., Ltd.) was used to detect and take pictures. The results were shown in FIG. 2 (a). The specifically expressed band was detected at 90 KD, which was larger than the theoretical molecular weight. This is mainly due to the modification of glycosylation sites on Fc when expressed in CHO-S cells, thereby increasing the molecular weight.

3.2 Screening and Verification of Positive Monoclonals

The cells after infected with lentivirus were resuspended with CD-SFM medium+5% FBS to 10 cells/mL. 100 μL of cell suspension was added to each well of a 96-well plate, cultured for 10 to 14 days, and then observed under a microscope about the formation of a monoclonal. According to the instructions of the human IgG ELISA quantitative kit (purchased from Beijing Daktronics Biotechnology Co., Ltd.), 50 μL of the cell culture supernatant was taken to detect protein expression. High expression cell lines were selected, and finally 2 high expression cell lines (1B7, 3E8) for each protein were obtained after screening. The test results are shown in Table 3.

TABLE 3
Monoclonal cell culture supernatant ELISA test results

| No. | OD450 | No. | OD450 | No. | OD450 |
|---|---|---|---|---|---|
| 1B3 | 0.0854 | 2B10 | 0.3062 | 3C5 | 0.7268 |
| 1B7 | 1.5640 | 2C2 | 0.4588 | 3C6 | 0.9519 |
| 1C11 | 0.5670 | 2C4 | 0.2153 | 3C9 | 0.2938 |
| 1D6 | 0.2386 | 2E5 | 0.1286 | 3D6 | 0.0461 |
| 1E2 | 0.0237 | 2E9 | 0.0533 | 3E8 | 1.3380 |
| 1G7 | 0.3485 | 2F3 | 0.9846 | 3F2 | 0.3794 |
| 1G2 | 0.6290 | 2G7 | 0.2654 | 3F11 | 0.8542 |
| | | 2G11 | 0.1084 | 3G3 | 0.0985 |

### Example 4 Purification and Quantification of SPHK1-Fc Protein

The monoclonal 1B7 was selected and delivered in a 500 mL shake flask to expand the culture. After centrifuged at 1200 rpm for 10 min, the cell pellet was removed, and the supernatant was collected. The supernatant was filtered with a 0.22 μm filter to remove cell debris. Protein A affinity column HiTrap MabSelect SuRe (purchased from GE General) was treated with 5 column volumes of equilibration buffer (5.6 mM NaH₂PO₄, 14.4 mM Na₂HPO₄, 0.15 M NaCl, pH 7.2), and then the supernatant was loaded. After loading, the loosely bound conjugate protein was washed with buffer (5.6 mM NaH₂PO₄.H₂O, 14.4 mM Na₂HPO₄, 0.5M NaCl, pH7.2) to Baseline. Then the protein was eluted with 50 mM citric acid/sodium citrate buffer (containing 0.02% Tween-80+5% mannitol, pH 3.2), and then was adjusted with 1M Tris-Cl (pH 8.0) to pH 7.0. The purified sample was filtered and sterilized through a 0.22 μm filter membrane and stored at 4° C.

The protein concentration of the purified sample was detected by using BCA protein quantification kit (purchased from Beijing Yuanpinghao Biotechnology Co., Ltd.). According to the quantitative results, 10 μg of protein was taken and subjected to SDS-PAGE electrophoresis with 10% gel, and stained and developed with a rapid protein staining kit (purchased from Beijing Yuanpinghao Biotechnology Co., Ltd.). The results were scanned and saved. As shown in FIG. 2(b), the size of the main protein obtained after purification is consistent with the result of immunoblotting in Example 3.1.

### Example 5 In Vivo Pharmacodynamic Study of SPHK1-Fc Protein

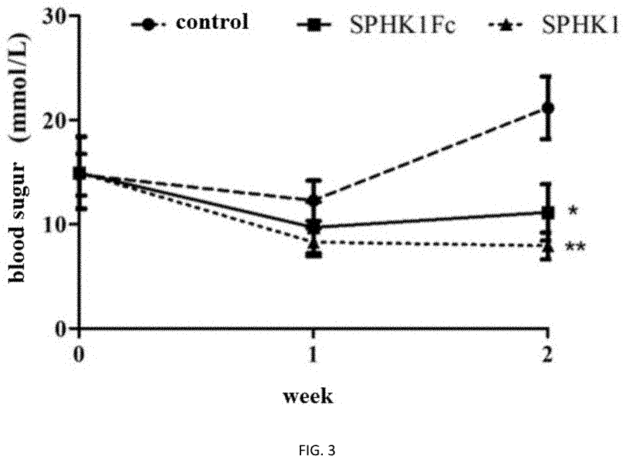

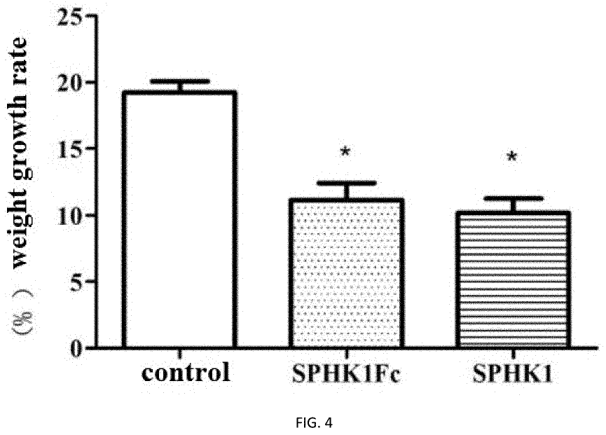

Eighteen 4-8 week old male type 2 diabetes model mice BKS.Cg-Dock7m+/+ Leprdb/Nju (purchased from the Institute of Animal Modeling, Nanjing University) were divided into three groups based on body weight and fasting blood glucose: control group (control, physiological saline), administration group 1 (SPHK1-Fc protein) and administration group 2 (SPHK1 protein, purchased from Beijing Yiqiao Shenzhou Biotechnology Co., Ltd., Catalog No. 15679-HNCB), 6 mice per group. The administration method of each group was subcutaneous injection, and the dosage was 2 mg/kg. The control group and the administration group 1 were administered twice a week, and the administration group 2 was administered once a day. The weight of the mice was weighed and recorded every week, and the fasting blood glucose of the mice was also measured. The mice were fasted for 12 hours on the night of the administration (normal water supply), and the blood glucose was measured the next morning. According to the average blood glucose of the mice, the blood glucose change curve was drawn. The results in FIG. 3 showed that the fasting blood glucose of the two administration groups was significantly lower than that of the control group after two weeks of treatment. The results in FIG. 4 showed that the weight of the mice in the two administration groups was also significantly lighter than the control group. However, the differences in fasting blood glucose and weight gain between the two administration groups had no statistical significance. It was demonstrated that SPHK1 and SPHK1-Fc have a significant effect in controlling blood sugar and body weight.

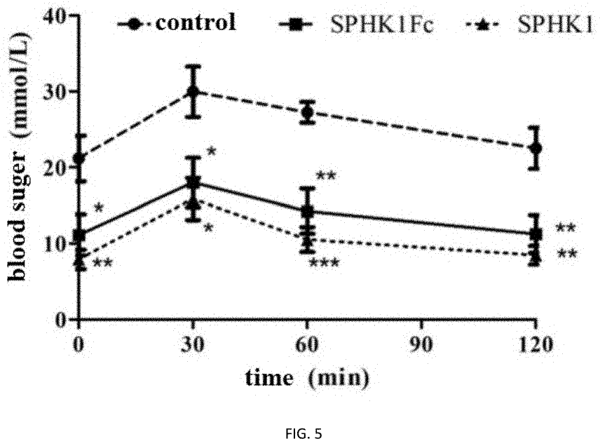

Glucose tolerance was measured after 2 weeks of treatment. Specifically, the mice were fasted for 12 hours the night before measured and then were injected intraperitoneally with glucose at a dose of 1 g glucose/kg, and blood glucose of mice was measured at 0, 30, 60 and 120 min. The results in FIG. 5 showed that the blood glucose levels of the two administration groups were lower than those of the control group at each test point, and there was no significant difference in glucose tolerance between the two administration groups, indicating that the mice had a significant improvement in glucose tolerance after administration.

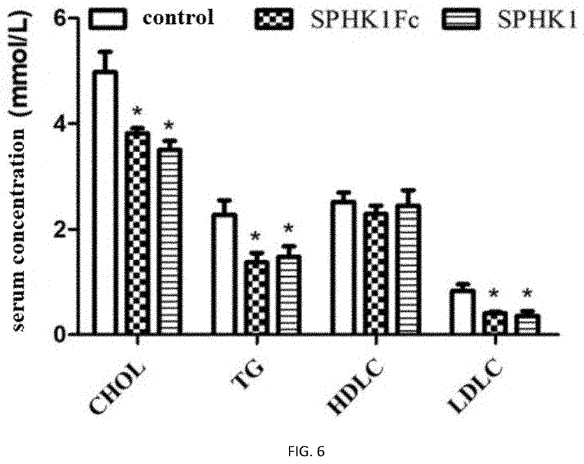

After 5 days of glucose tolerance test, serum biochemical indicators were detected. Specifically, blood was taken from mouse eyeballs and centrifuged at 3000 rpm for 10 minutes to separate serum, and samples were sent to Beijing North Biotech Medical Technology Co., Ltd. for detection of triglyceride (TG), total cholesterol (CHOL), high density lipoprotein (HDLC) and low density lipoprotein (LDLC). The results in FIG. 6 showed that after SPHK1-Fc and SPHK1 protein treatment, the levels of CHOL, TG and LDLC in mice were significantly lower than those in the control group, indicating that SPHK1-Fc and SPHK1 had a regulatory effect on blood lipid metabolism and can effectively control blood lipid levels.

The present invention has been described in detail in combination with embodiments and examples. However, it should be noted that these embodiments are merely illustrative for the present invention and do not constitute any limitation to the scope of the present invention. Within the spirit and the scope of the present invention, various improvements, equivalent substitutions or modifications can be made to the technical content of the present invention and its embodiments, all of which fall in the scope of the present invention.

What is claimed is:

1. A method for preventing and/or treating a disease comprises a step of administrating a subject in need with a sphingosine kinase 1 or an amino acid sequence having the activity thereof, wherein the disease is selected from the group consisting of obesity, hyperlipidemia and diabetes; the sphingosine kinase 1 or the amino acid sequence having the activity thereof comprises a protein having the amino acid sequence shown in SEQ ID NO:1.

2. A protein drug, comprising a sphingosine kinase 1 or an amino acid sequence having the activity thereof, wherein the protein drug is a fusion protein containing the sphingosine kinase 1 or the amino acid sequence having its activity.

3. The protein drug according to claim 2, wherein the fusion protein contains the sphingosine kinase 1 (SPHK1) or the amino acid sequence having the activity thereof, a FC sequence and a linker sequence;
the FC sequence is selected from the amino acid sequence of human or animal immunoglobulin and its subtypes and variants, or the amino acid sequence of human or animal albumin and its variants;
a general formula of the linker sequence is (GGGGS)n, where n is an integer of 0-5;
preferably, the human or animal immunoglobulin is selected from IgG4FC fragments;
more preferably, the human or animal immunoglobulin is a peptide having the amino acid sequence shown in SEQ ID NO: 12;
the fusion protein contains the amino acid sequence shown in SEQ ID NO:2.

4. The protein drug according to claim 2, wherein the fusion protein is modified with a polyethylene glycol; wherein the average molecular weight of the polyethylene glycol is 5-50 KD.

5. The protein drug according to claim 2, wherein the protein drug is encoded by a nucleotide having the nucleotide sequence shown in SEQ ID NO:3.

6. The protein drug according to claim 2, wherein a nucleotide having the nucleotide sequence shown in SEQ ID NO:3 is constructed into a prokaryotic expression vector or a eukaryotic expression vector.

7. The protein drug according to claim 6, wherein the prokaryotic expression vector is a pET vector;
the eukaryotic expression vector is selected from the group consisting of pVAX1 vector, pSV1.0 vector, a recombinant virus vector, a recombinant vaccinia virus vector, a recombinant adenovirus vector, a recombinant adeno-associated virus vector, a retroviral vector/lentiviral vector and a HIV viral vector.

8. The protein drug according to claim 6, wherein expression vector is transduced into a host cell
when the expression vector is the prokaryotic expression vector, the host cell is a prokaryotic cell, preferably bacterial cell;
when the expression vector is a eukaryotic expression vector, the host cell is a eukaryotic cell, preferably mammalian cell, and more preferably CHO cell.

9. A method for preparing a protein drug, comprising a step of cloning a nucleotide sequence being capable of encoding the protein drug claim 2 into an expression vector, wherein the method comprises the steps as follows:
1) constructing the nucleic acid sequence of the protein drug;
2) constructing the expression vector containing the nucleic acid sequence of step 1);
3) utilizing the expression vector of step 2) to transfect or transform a host cell and allow the nucleic acid sequence to be expressed in the host cell;
and preferably, in step 3), the host cell is a CHO-S cell.

10. The protein drug according to claim 6, wherein the host cell is used for preparing a pharmaceutical composition for preventing and/or treating obesity, hyperlipidemia or diabetes.
